SEQUENCE LISTING (38 sequences)

SEQ ID NO: 1 - DNA, 54 bp, Artificial Sequence
  Description of Artificial Sequence Synthetic primer
aacctacacaaagtttctgctaacgatctctcggtgtttcgtcctttccacaag

SEQ ID NO: 2 - DNA, 54 bp, Artificial Sequence
  Description of Artificial Sequence Synthetic primer
cggcgaagctttttccaaaaaaagagatcgttagcagaaacctacacaaagttt

SEQ ID NO: 3 - DNA, 55 bp, Artificial Sequence
  Description of Artificial Sequence Synthetic primer
tgtctacacaaaacaaactgaggattgcaagccggtgtttcgtcctttccacaag

SEQ ID NO: 4 - DNA, 54 bp, Artificial Sequence
  Description of Artificial Sequence Synthetic primer
cggcgaagctttttccaaaaaacttgcaatcctcagtttgtctacacaaaacaa

SEQ ID NO: 5 - DNA, 55 bp, Artificial Sequence
  Description of Artificial Sequence Synthetic primer
tgtctacacaaaacatagtgaagataggaagccggtgtttcgtcctttccacaag

SEQ ID NO: 6 - DNA, 54 bp, Artificial Sequence
  Description of Artificial Sequence Synthetic primer
cggcgaagctttttccaaaaaacttcctatcttcactatgtctacacaaaacat

SEQ ID NO: 7 - DNA, 54 bp, Artificial Sequence
  Description of Artificial Sequence Synthetic primer
acactacacaaatgtttttgtaaagtatagtcggtgtttcgtcctttccacaag

SEQ ID NO: 8 - DNA, 54 bp, Artificial Sequence
  Description of Artificial Sequence Synthetic primer
cggcgaagctttttccaaaaaaactatactttacaaaaacactacacaaatgtt

SEQ ID NO: 9 - DNA, 54 bp, Artificial Sequence
  Description of Artificial Sequence Synthetic primer
aaactacacaaatttccagctttacagtgaaccggttttcgtcctttccacaag

SEQ ID NO: 10 - DNA, 54 bp, Artificial Sequence
  Description of Artificial Sequence Synthetic primer
cggcgaagctttttccaaaaaattcactgtaaagctggaaactacacaaatttc

SEQ ID NO: 11 - DNA, 55 bp, Artificial Sequence
  Description of Artificial Sequence Synthetic primer
gggctacacaaaccctacgtgaatcggattgccggtgtttcgtcctttccacaag

SEQ ID NO: 12 - DNA, 54 bp, Artificial Sequence
  Description of Artificial Sequence Synthetic primer
cggcgaagctttttccaaaaaacaatccgattcacgtagggctacacaaaccct

SEQ ID NO: 13 - DNA, 54 bp, Artificial Sequence
  Description of Artificial Sequence Synthetic primer
atcctacacaaagatgaacttcagggtcagccggtgtttcgtcctttccacaag

SEQ ID NO: 14 - DNA, 54 bp, Artificial Sequence
  Description of Artificial Sequence Synthetic primer
cggcgaagctttttccaaaaaagctgaccctgaagttcatcctacacaaagatg

SEQ ID NO: 15 - DNA, 54 bp, Artificial Sequence
  Description of Artificial Sequence Synthetic primer
aacctacacaaagttgtagttgtactccagccggtgtttcgtcctttccacaag

SEQ ID NO: 16 - DNA, 54 bp, Artificial Sequence
  Description of Artificial Sequence Synthetic primer
cggcgaagctttttccaaaaaagctggagtacaactacaacctacacaaagttg

SEQ ID NO: 17 - DNA, 55 bp, Artificial Sequence
  Description of Artificial Sequence Synthetic primer
aacctacacaaagttgtggcggatcttgaagccggtgtttcgtcctttccacaag

SEQ ID NO: 18 - DNA, 54 bp, Artificial Sequence
  Description of Artificial Sequence Synthetic primer
cggcgaagctttttccaaaaaacttcaagatccgccacaacctacacaaagttg

SEQ ID NO: 19 - DNA, 26 bp, Artificial Sequence
  Description of Artificial Sequence Synthetic primer
cccaagcttagttattaatagtaatc

SEQ ID NO: 20 - DNA, 28 bp, Artificial Sequence
  Description of Artificial Sequence Synthetic primer
ggaagatcttacttgtacagctcgtcca

SEQ ID NO: 21 - DNA, 26 bp, Artificial Sequence
  Description of Artificial Sequence Synthetic primer
ggaagatcttaatcagccataccaca

SEQ ID NO: 22 - DNA, 28 bp, Artificial Sequence
  Description of Artificial Sequence Synthetic primer
ccggaattcacgcgttaagatacattga

SEQ ID NO: 23 - DNA, 26 bp, Artificial Sequence
  Description of Artificial Sequence Synthetic primer
cccaagcttagttattaatagtaatc

SEQ ID NO: 24 - DNA, 28 bp, Artificial Sequence
  Description of Artificial Sequence Synthetic primer
ggaagatcttacttgtacagctcgtcca

SEQ ID NO: 25 - DNA, 18 bp, Artificial Sequence
  Description of Artificial Sequence Synthetic primer
tcaggctgcgcaactgtt

SEQ ID NO: 26 - DNA, 19 bp, Artificial Sequence
  Description of Artificial Sequence Synthetic primer
tgagttagctcactcatta

SEQ ID NO: 27 - DNA, 22 bp, Artificial Sequence
  Description of Artificial Sequence Synthetic primer
cggaattcgatccgacgccgcc

SEQ ID NO: 28 - DNA, 22 bp, Artificial Sequence
  Description of Artificial Sequence Synthetic primer
cggcgaagctttttccaaaaaa

SEQ ID NO: 29 - DNA, 27 bp, Artificial Sequence
  Description of Artificial Sequence Synthetic primer
cccaagcttgctcccagacatgacagc

SEQ ID NO: 30 - DNA, 32 bp, Artificial Sequence
  Description of Artificial Sequence Synthetic primer
agctttgtttaaactcagacttttgtaatttg

SEQ ID NO: 31 - DNA, 22 bp, Artificial Sequence
  Description of Artificial Sequence Synthetic primer
tcggcaaggtgatcctggtgaa

SEQ ID NO: 32 - DNA, 22 bp, Artificial Sequence
  Description of Artificial Sequence Synthetic primer
aggcggtcgtgggtctggaaag

SEQ ID NO: 33 - DNA, 22 bp, Artificial Sequence
  Description of Artificial Sequence Synthetic primer
cggccacaagttcagcgtgtcc

SEQ ID NO: 34 - DNA, 22 bp, Artificial Sequence
  Description of Artificial Sequence Synthetic primer
cggggtagcggctgaagcactg

SEQ ID NO: 35 - DNA, 20 bp, Artificial Sequence
  Description of Artificial Sequence Synthetic primer
ccccccgtggcggcgacgac

SEQ ID NO: 36 - DNA, 20 bp, Artificial Sequence
  Description of Artificial Sequence Synthetic primer
ttgccctccaatggatcctc

SEQ ID NO: 37 - DNA, 22 bp, Artificial Sequence
  Description of Artificial Sequence Synthetic primer
gacaggattgacagattgatag

SEQ ID NO: 38 - DNA, 22 bp, Artificial Sequence
  Description of Artificial Sequence Synthetic primer
cacttgtccctctaagaagttg

(19) United States
(12) Patent Application Publication
(10) Pub. No.: US 2007/0135368 A1
(43) Pub. Date: Jun. 14, 2007

(54) Cell-to-cell transmission of siRNA induced gene silencing in mammalian cells

(76) Inventors: Pamela E. Knapp, Lexington, KY (US); Tianyong Zhao, Lexington, KY (US); Shi-Ping Zhao, Nicholasville, KY (US); Yelena V. Alimova, Lexington, KY (US); Guoying Wang, Haidian (CN); Kurt F. Hauser, Lexington, KY (US); Mohammed Said Ghandour, Strasbourg (FR)

Correspondence Address: BUCHANAN, INGERSOLL & ROONEY PC, POST OFFICE BOX 1404, ALEXANDRIA, VA, 22313-1404, US

(21) Appl. No.: 11/352,513

(22) Filed: Feb. 10, 2006

Related U.S. Application Data
(60) Provisional application No. 60/748,582, filed on Dec. 9, 2005.

(51) Int. Cl. A61K 48/00; C12N 5/08

(52) U.S. Cl. 514/44; 435/368; 435/455

(57) ABSTRACT

A method for reducing expression of a target gene in a mammalian cell is provided. The method comprises (a) introducing siRNA into a first mammalian cell such that expression of the target gene is reduced in the first mammalian cell, and (b) exposing a second mammalian cell which does not contain siRNA that reduces expression of the target gene to the siRNA-containing first mammalian cell, thereby resulting in reduced expression of the target gene in the second mammalian cell. An additional aspect provides a method of reducing expression of a target gene in a mammal. Yet another aspect provides a mammalian cell transfected with an expression vector which directs expression of siRNA for a target gene such that expression of the target gene is reduced in the transfected mammalian cell, and the siRNA expression level is sufficient to signal reduction of target gene expression in a mammalian cell not transfected with siRNA for the target gene.

### CROSS REFERENCE TO RELATED APPLICATIONS

This application claims priority to U.S. Provisional Application Ser. No. 60/748,582 filed Dec. 7, 2005, the disclosure of which is incorporated herein by reference.

### FIELD OF THE INVENTION

The present invention relates to the cell-to-cell transfer of gene silencing in mammalian cells.

### DESCRIPTION OF THE RELATED ART

Double-stranded RNA (dsRNA) triggers sequence-specific RNA degradation and causes RNA silencing (Fire et al., Nature, 391:806-811,1998). This phenomenon is called RNA interference or RNAi. In plants and C. elegans, RNAi can be induced locally and then spread to distant sites throughout the organism, or transmitted to the next generation (Palauqui et al., EMBO J., 16:4738-4745, 1997; Voinnet et al., Nature, 389:553, 1997; Voinnet et al., Cell, 95:177-187, 1998; Mlotshwa et al., Plant Cell, 14(suppl): s289-s301, 2002; Hamilton et al, EMBO J., 21:4671-4679, 2002; Garcia-Perez et al., Plant J., 38:594-602, 2004; Himber et al., EMBO J., 22:4523-4533, 2003; Grishok et al., Science, 287:2494-2497, 2000). There has been no indication of siRNA replication or cell to cell transmission of silencing in mammalian cells.

### SUMMARY OF THE INVENTION

In one embodiment the invention provides a method of reducing expression of a target gene in a mammalian cell. The method comprises (a) introducing siRNA into a first mammalian cell such that expression of the target gene is reduced in the first mammalian cell, and (b) exposing a second mammalian cell which does not contain siRNA that reduces expression of the target gene to the siRNA-containing first mammalian cell, thereby resulting in reduced expression of the target gene in the second mammalian cell. In one aspect the method involves introducing siRNA into the first mammalian cell by transfection with an expression vector.

In another embodiment the invention provides a method of reducing expression of a target gene in a mammal. The method comprises (a) introducing siRNA into a first mammalian cell such that expression of the target gene is reduced in the first mammalian cell, and (b) introducing the first mammalian cell containing the siRNA into the mammal whereby expression of the target gene is reduced in a cell of the mammal. In one aspect the method involves introducing siRNA into the first mammalian cell by transfection with an expression vector.

In yet another embodiment, the invention provides a transfected mammalian cell wherein the cell is transfected with an expression vector which directs expression of siRNA for a target gene such that expression of the target gene is reduced in the transfected mammalian cell, and the siRNA expression level is sufficient to signal reduction of target gene expression in a mammalian cell not transfected with siRNA for the target gene.

Other systems, methods, features and advantages of the present invention will be or become apparent to one with skill in the art upon examination of the following drawings and detailed description. It is intended that all such additional systems, methods, features and advantages be included within this description, be within the scope of the present invention, and be protected by the accompanying claims.

### BRIEF DESCRIPTION OF THE DRAWINGS

FIGS. 1A, 1B, 1C, 1D, 1E, 1F, 1G and 1H set forth data showing PTEN or AKT silencing can be transmitted among cultured mammalian cells.

FIGS. 2A and 2B set forth data on testing the efficacy of PTEN-SECs.

FIGS. 3A, 3B and 3C set forth data showing the effects of transfection of cells with expression vectors for GFP or PTEN siRNA.

FIGS. 4A, 4B and 4C set forth data showing that GFP silencing can be transmitted among cultured mammalian cells.

FIG. 5 illustrates real time RT-PCR analysis of PTEN expression in co-cultures.

### DESCRIPTION OF PREFERRED EMBODIMENTS

Any mammalian cell can be used according to the present invention. These mammalian cells include, by way of example, neural cells, epithelial cells, epidermal cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc. Moreover, the mammalian cells used may be obtained from different organs, e.g., brain, skin, lung, pancreas, liver, stomach, intestine, esophagus, spleen, thymus, thyroid gland, salivary gland, bone, heart, skeletal muscle, reproductive organs, bladder, kidney, urethra and other urinary organs, etc. Cells which can be used include all somatic or germ cells. Neural cells are preferred.

Neural cells which can be used according to the invention include oligodendrocytes, neuronal cells, glial cells, astrocytes, and neuronal restricted precursors. Cancerous nervous cells, such as glioma cells, also can be used.

Cells from any mammalian species can be used according to the invention, including human, monkey, cow, sheep, pig, goat, mouse, rat, dog, cat, rabbit, guinea pig, hamster and horse. Human cells are preferred.

Methods of the invention for reducing expression of a target gene can be performed in vitro, in vivo or ex vivo. For example, siRNA can be introduced into a mammalian cell in culture such that expression of the target gene is reduced. The siRNA-containing cell can then be exposed to a second mammalian cell in culture such that expression of the target gene is reduced in the second mammalian cell. In another aspect, the siRNA-containing cell can be introduced into a mammal such that expression of the target gene is reduced in one or more cells of the mammal. In yet another aspect, siRNA can be introduced into a first cell in a mammal such that expression of the target gene is reduced. The siRNA-containing cell can then be exposed to a second cell in the mammal such that expression of the target gene is reduced in the second mammalian cell

The siRNAs for use in the present invention are designed according to standard methods in the field of RNA interference. Introduction of siRNAs into cells may be by transfection with expression vectors, by transfection with synthetic dsRNA, or by any other appropriate method. See, for example, Sui et al., Proc. Natl. Acad. Sci. USA, 99:5515-5520, 2002; and Elbashir et al., Nature, 411:494-498, 2001. Transfection with expression vectors is preferred.

The expression vectors which can be used to deliver siRNA according to the invention include retroviral, adenoviral and lentiviral vectors. The expression vector includes a sequence which codes for a portion of the target gene which is to be silenced. The target gene sequence is designed such that, upon transcription in the transfected host, the target RNA sequence forms a hairpin structure due to the presence of self-complementary bases. Processing within the cell removes the loop resulting in formation of a siRNA duplex. The double stranded RNA sequence should be less than 30 nucleotide bases; preferably the dsRNA sequence is 19-25 bases in length; more preferably the dsRNA sequence is 19, 20 or 21 nucleotides in length.

The expression vectors may include one or more promoter regions to enhance synthesis of the target gene sequence. Promoters which can be used include CMV promoter, SV40 promoter, promoter of mouse U6 gene, and promoter of human H1 gene.

One or more selection markers may be included to facilitate transfection with the expression vector. The selection marker may be included within the expression vector, or may be introduced on a separate genetic element. For example, the bacterial hygromycin B phosphotransferase gene may be used as a selection marker, with cells being grown in the presence of hygromycin to select for those cells transfected with the aforementioned gene.

Synthetic dsRNA may also be introduced into cells to provide gene silencing by siRNA. The synthetic dsRNAs are less than 30 base pairs in length. Preferably the synthetic dsRNAs are 19-25 base pairs in length. More preferably the dsRNAs are 19, 20 or 21 base pairs in length, optionally with 2-nucleotide 3′ overhangs. The 3′ overhangs are preferably TT residues.

Synthetic dsRNAs can be introduced into cells by injection, by complexing with agents such as cationic lipids, by use of a gene gun, or by any other appropriate method.

The methods of the invention can be used to treat various diseases and disorders. For example, the target gene whose expression is to be reduced may be a mutant gene which causes a genetic disease, such as Huntington's disease or Pelizaeus-Merzbacher disease. In another embodiment, the target gene is a gene whose overexpression causes a disease, such as a cancer. Such cancers include, for example, a cancer in which the EGF-receptor is overexpressed. Additional examples of such cancers include cancers of the central nervous system, such as a glioblastoma. Another disease caused by overexpression of a target gene is Pelizaeus-Merzbacher disease in which the proteolipid protein gene is overexpressed. In other embodiments, the target gene is a death gene which causes loss of a cell after a traumatic injury, or the target gene is an MHC gene whose absence increases the vulnerability of a tumor cell to NK mediated killing. In yet another embodiment, the target gene is a gene which promotes cell survival such that expression of the siRNA induces cell death, e.g., such as where the cell death occurs in a tumor cell or an astroglial scar.

Plasmid Construction

Plasmid pEGFP-C1 was purchased from Becton Dickinson (Palo Alto, Calif.). Plasmid pSEC™hygro was purchased from Ambion (Austin, Tex.). The DNA fragment containing the CMV promoter driving the GFP coding region was amplified by PCR from plasmid pEGFP-C1 using primer pair 1 (Table 1). SV4O poly-A fragment was amplified from plasmid pEGFP-C1 using primer pair 2 (Table 1). Restriction sites for Hind III, Bgl II or EcoR I were included in each primer to facilitate cloning. PCR products were purified using a PCR purification kit (Qiagen, Valencia, Calif.), digested with Hind III and Bgl II or Bgl II and EcoR I and then gel purified. The purified fragments were ligated together and inserted into the corresponding sites of the pSEC™hygro vector to form a GFP expressing vector which also contained the hygromycin selectable marker gene. siRNA expression cassettes (SEC) for PTEN (phosphatase and tensin homolog deleted on chromosome 10), AKT (Stall, Proc. Natl. Acad. Sci. U.S.A., 84(14):5034-5037, 1987), GFP (green fluorescent protein), non-specific negative control and GAPDH genes were prepared using the mouse U6 gene promoter or human H1 gene promoter to drive a gene specific or non specific hairpin DNA according to the Silencer™ Express Kit (Ambion) protocol. siRNA targets for PTEN, AKT or GFP genes were selected according to their cDNA sequences (GenBank# 1916329, 62241014 and U55763) using online software (siRNA Target Finder) from Ambion, and were determined to be non-homologous to other genes by BLAST research. Sense and antisense primers (Table 1) were designed based on target sequences using the same software. Sense and antisense primers for non-specific negative control and GAPDH gene were provided by the Ambion kit. The SECs were cloned into pSEC™hygro vector or directly used for transfection.

TABLE 1
Primer List

| | |
|---|---|
| Primer | Sequence (5′-3′) |

| | |
|---|---|
| PTEN 1 sense | AACCTACACAAAGTTTCTGCTAACGATCTCTC |
| | GGTGTTTCGTCCTTTCCACAAG |
| PTEN 1 antisense | CGGCGAAGCTTTTTCCAAAAAAAGAGATCGTT |
| | AGCAGAAACCTACACAAAGTTT |
| PTEN 2 sense | TGTCTACACAAAACAAACTGAGGATTGCAAGC |
| | CGGTGTTTCGTCCTTTCCACAAG |
| PTEN 2 antisense | CGGCGAAGCTTTTTCCAAAAAACTTGCAATCC |
| | TCAGTTTGTCTACACAAAACAA |
| PTEN 2m sense | TGTCTACACAAAACATAGTGAAGATAGGAAGC |
| | CGGTGTTTCGTCCTTTCCACAAG |
| PTEN 2m antisense | CGGCGAAGCTTTTTCCAAAAAACTTCCTATCT |
| | TCACTATGTCTACACAAAACAT |
| PTEN 3 sense | ACACTACACAAATGTTTTTGTAAAGTATAGTC |
| | GGTGTTTCGTCCTTTCCACAAG |
| PTEN 3 antisense | CGGCGAAGCTTTTTCCAAAAAAACTATACTTT |
| | ACAAAAACACTACACAAATGTT |
| PTEN 4 sense | AAACTACACAAATTTCCAGCTTTACAGTGAAC |
| | CGGTTTTCGTCCTTTCCACAAG |
| PTEN 4 antisense | CGGCGAAGCTTTTTCCAAAAAATTCACTGTAA |
| | AGCTGGAAACTACACAAATTTC |
| AKT sense | GGGCTACACAAACCCTACGTGAATCGGATTGC |
| | CGGTGTTTCGTCCTTTCCACAAG |
| AKT antisense | CGGCGAAGCTTTTTCCAAAAAACAATCCGATT |
| | CACGTAGGGCTACACAAACCCT |
| GFP 1 sense | ATCCTACACAAAGATGAACTTCAGGGTCAGCC |
| | GGTGTTTCGTCCTTTCCACAAG |
| GFP 1 antisense | CGGCGAAGCTTTTTCCAAAAAAGCTGACCCTG |
| | AAGTTCATCCTACACAAAGATG |
| GFP 2 sense | AACCTACACAAAGTTGTAGTTGTACTCCAGCC |
| | GGTGTTTCGTCCTTTCCACAAG |
| GFP 2 antisense | CGGCGAAGCTTTTTCCAAAAAAGCTGGAGTAC |
| | AACTACAACCTACACAAAGTTG |
| GFP 3 sense | AACCTACACAAAGTTGTGGCGGATCTTGAAGC |
| | CGGTGTTTCGTCCTTTCCACAAG |
| GFP 3 antisense | CGGCGAAGCTTTTTCCAAAAAACTTCAAGATC |
| | CGCCACAACCTACACAAAGTTG |
| Primer 1 forward | CCCAAGCTTAGTTATTAATAGTAATC |
| Primer 1 reverse | GGAAGATCTTACTTGTACAGCTCGTCCA |
| Primer 2 forward | GGAAGATCTTAATCAGCCATACCACA |
| Primer 2 reverse | CCGGAATTCACGCGTTAAGATACATTGA |
| Primer 3 forward | CCCAAGCTTAGTTATTAATAGTAATC |
| Primer 3 reverse | GGAAGATCTTACTTGTACAGCTCGTCCA |
| Primer 4 forward | TCAGGCTGCGCAACTGTT |
| Primer 4 reverse | TGAGTTAGCTCACTCATTA |
| Primer 5 forward | CGGAATTCGATCCGACGCCGCC |
| Primer 5 reverse | CGGCGAAGCTTTTTCCAAAAAA |
| Primer 6 forward | CCCAAGCTTGCTCCCAGACATGACAGC |
| Primer 6 reverse | AGCTTTGTTTAAACTCAGACTTTTGTAATTTG |
| Primer 7 forward | TCGGCAAGGTGATCCTGGTGAA |
| Primer 7 reverse | AGGCGGTCGTGGGTCTGGAAAG |
| Primer 8 forward | CGGCCACAAGTTCAGCGTGTCC |
| Primer 8 reverse | CGGGGTAGCGGCTGAAGCACTG |
| Primer 9 forward | CCCCCCGTGGCGGCGACGAC |
| Primer 9 reverse | TTGCCCTCCAATGGATCCTC |
| Primer 10 forward | GACAGGATTGACAGATTGATAG |
| Primer 10 reverse | CACTTGTCCCTCTAAGAAGTTG |

Astrocyte Culture

Primary astrocyte cultures are prepared from postnatal day 0-2 mouse pups. Cerebral cortices are aseptically removed and cleaned of meninges. Brain tissue is diced in serum free medium and then enzymatically (trypsin/DNAse, 37° C., 30 mm) and mechanically dissociated, filtered through nylon mesh and plated in Dulbecco's Modified Eagle's Medium with 10% fetal calf serum (HyClone, Logan, Utah) and antibiotics at low density (1 brain/T-75 flask) on poly-L-lysine (Sigma, St. Louis, Mo.) coated plasticware. Cells are split once when confluent (˜8 days) and plated in 6 well plates (5×10⁵ cells/well). Unless noted, all culture materials were purchased from Invitrogen (Gaithersburg, Md.).

Cell Cultures and Transfections

Immortalized 158N OLs (oligodendrocytes), primary astrocytes and SNB19 cells were cultured in Dulbecco's Modified Eagle's Medium supplemented with 0.5% glucose, 100 units/ml penicillin-streptomycin and 10% characterized fetal bovine serum (HyClone, Logan, Utah) at 37° C., 5% CO₂. Cells were transfected in 6-well plates using the Ambion siPORT XP-1 transfection reagent. All transfection procedures followed the manufacturer's recommended protocol. The amount of siPORT XP-1 and DNA was optimized for higher transfection efficiency based on GFP expression. For one well of cells (5×10⁵ cells), 4.8 μl of siPORT XP-1 was mixed with 200 μl of media and 2 μg of DNA. For selection of stable transfected cells, hygromycin B was added to the media 2 days after transfection at a final concentration of 250 μg/ml. Resistant colonies appeared after 10 days. Single colonies were selected using sterile cloning discs (PGC Scientific, Frederick, Md.).

Western Blot Analysis

Total protein was extracted using RIPA buffer (50 mM Tris pH 7.4, 150 mM NaCl, 1% Nonidet, 0.5% deoxycholic acid, 0.1% SDS, one tablet of protease inhibitors from Roche in 10 ml RIPA). Protein concentration was measured using the BCA Protein Assay Kit (Pierce Inc., Rockford, Ill.). For PTEN Western blots, 20 μg of total protein was separated on 15% Tris-HCl Ready Gels (Bio-Rad, Hercules, Calif.) and blotted to Hybond-P membrane (Amersham Biosciences, Piscataway, N.J.). Polyclonal antibody to PTEN was purchased from Upstate Biotechnology, Inc. (Lake Placid, N.Y.). Polyclonal GFP antibody was purchased from Research Diagnostics, Inc. (Flanders, N.J.). Actin monoclonal antibody was purchased from Chemicon International (Temecula, Calif.). Blots were detected using the ECL-Plus detection system (Amersham) and visualized on a Kodak Image Station 440 using ID Software.

PCR Analysis of Stably Transfected Cell Lines

Genomic DNA was isolated from cultured cells using DNeasy Tissue Kit (Qiagen). PCR was carried out using genomic DNA as template and using three pairs of primers to amplify the fragment of CMV-GFP, Hpt coding region and SECs of PTEN or negative control (primer pairs 3, 4, and 5, Table 1). PCR products were visualized by ethidium bromide staining in agarose gels.

Co-Cultures and Transwell Cultures

Co-cultures were plated in 60 mm² culture dishes. The two types of cells were mixed at different ratios as indicated in the figure legend and plated at a density of 4×10⁶ cells per dish. Cells were harvested after 3 days of co-culture to detect the target gene expression.

PTEN-SEC transfected 158N cells were co-cultured with 158N^{NCsi} cells (1:1 ratio) in 6-well plates at a density of 1×10⁶ cells per well. Cells were cultured for 2 days and then transferred to individual 100 mm petri-dishes to eliminate PTEN-SEC transfected cells using hygromycin selection (250 μg/ml medium). In co-cultures employing transwell plates, 158N^{Psi} cells (0.5×10⁶ cells) were grown in the transwell insert in the upper compartment while 158N non-silenced cells (0.5×10⁶ cells) were plated in the lower compartment well. Cells were harvested after 3 days of culture.

158N cells transiently transfected with either GFP-SEC, CMV-GFP, or with both constructs, were co-cultured with 158N^{GFP} cells (10:1 ratio) in 6-well plates at a density of 2×10⁶ cells per well. Cells were cultured for 2 days and then transferred to individual 60 mm² petri-dishes to eliminate the transiently transfected 158N cells using hygromycin selection (250 μg/ml medium).

FACS Sorting

Cultured cells were treated with cell dissociation buffer (Sigma) for 20 minutes and then collected by centrifugation. The cells were suspended in sterile sort buffer (1×PBS, Ca/Mg free; 1 mM EDTA; 25 mM HEPES, pH 7; 1% FBS) at a density of 10⁷ cells/ml. Cell suspensions were filtered through 70 μm nylon filter, transferred to 5 ml polypropylene tubes and sorted using excitation and emission wavelengths of 488 nm and 525 nm, respectively.

RNA Isolation and Analysis

Total RNA was isolated using GenElute™ Mammalian Total RNA kit (Sigma). cDNA was synthesized from 2 μg of total RNA using the Protoscript™ kit from Biolabs (Beverly, Mass.) or the High-Capacity cDNA Archive Kit (Applied Biosystems, Warrington, UK). RT-PCR was carried out using cDNA as template and using PTEN gene specific primers (primer pair 6, Table 1). Amplicons were separated on agarose gels, stained with ethidium bromide and photographed. Real time RT-PCR was also used to determine the PTEN, AKT or GFP expression. Standards containing varying concentrations of cDNA templates (cDNA mix and its 2×, 4×, 8×, 16× dilutions) were prepared to generate a standard curve. TaqMan Universal PCR Master Mixes, SYBR green mixture and Gene-specific TaqMan probe and primer sets for PTEN and eukaryotic 18S rRNA for quantitative gene expression assay were purchased from Applied Biosystems. The primers for AKT (primer pair 7, Table 1), GFP (primer pair 8, Table 1) and 18S (primer pair 9, Table 1) used in real time RT-PCR were designed based on their cDNA sequence. Real time RT-PCR was conducted using the Applied Biosystems Prism 7500 system. mRNA levels of PTEN, AKT and GFP were normalized against 18S mRNA. Northern hybridization followed a published protocol (Zhao et al., Plant Sci., 165:245-256, 2003). The probe templates for PTEN and 18S were obtained by RT-PCR using RNA isolated from 158N cells as template and using their gene specific primers (PTEN: primer pair 6; 18s: primer pair 10, Table 1).

Cell Cycle Analysis By Propidium Iodide DNA Staining

Cells grown nearly to confluent in 100 mm² petri dishes were washed with HBSS (pH 7.4) and harvested by trypsinization. Cell pellets were washed with ice-cold 1×PBS and diluted to a density of 2×10⁶ cells/ml. 5 ml of ice cold 70% ethanol was added dropwise to 0.5 ml of cells while vortexing to prevent clumping. Cells were kept at 4° C. for 20 min, pelleted (1,500 rpm, 5 mm), washed with cold PBS and resuspended in 0.5 ml of staining buffer (5 μg/ml of propidium iodide, 1 mg/ml of RNase A in PBS). Cell suspensions were incubated at 37° C. for 30 min prior to FACS analysis.

Rate of Cell Production

Both 158N^{GFP} cells and 158N^{Psi} cells were plated in 6 well plates at a density of 0.2×10⁶ cells per well with 2 ml of medium containing fetal bovine serum. Cells in individual wells were harvested and counted after 3 days. The relative rate of cell production was calculated as the final cell number divided by the initial cell number.

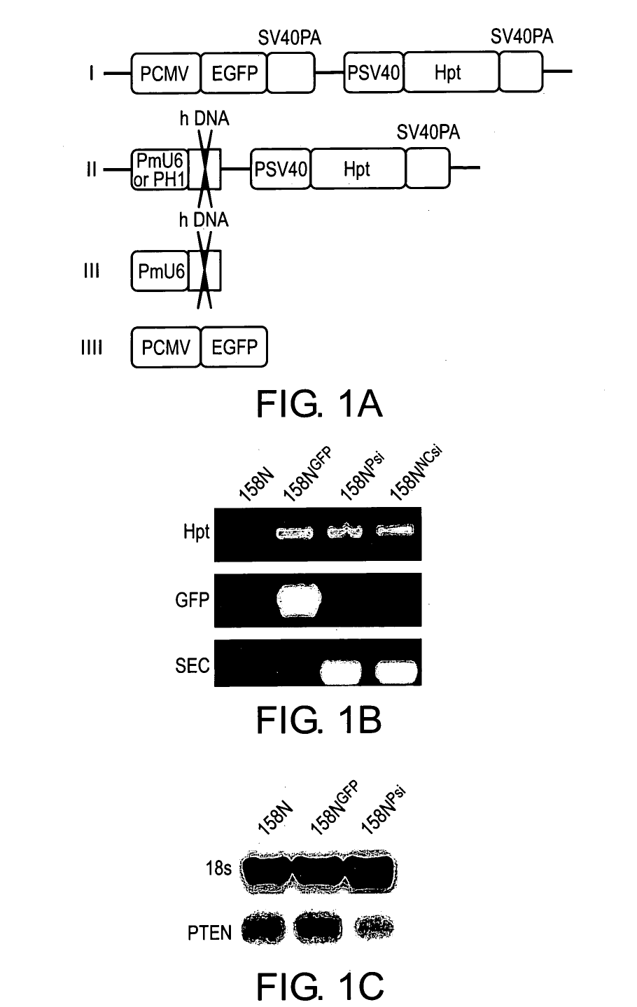

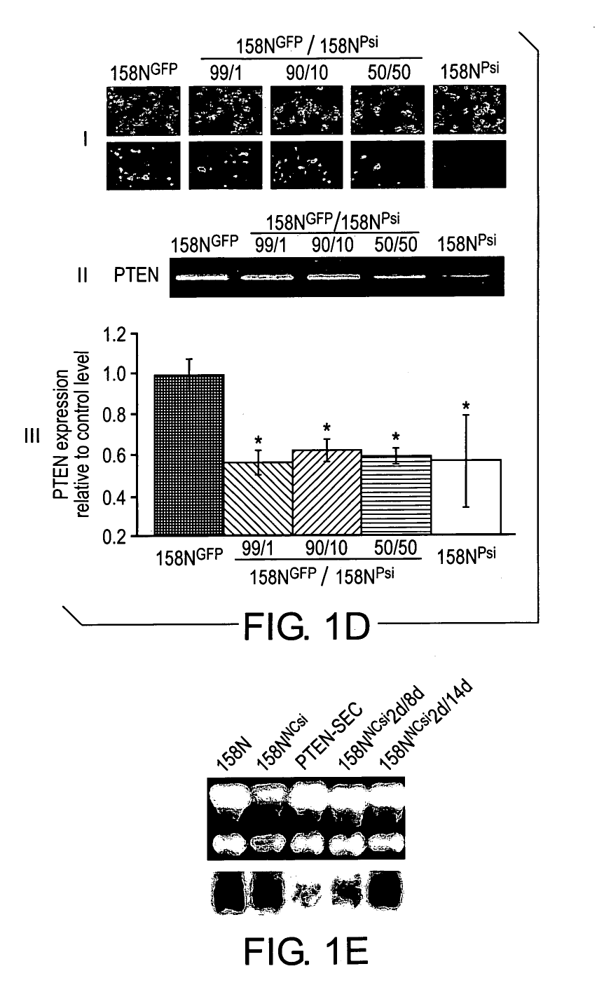

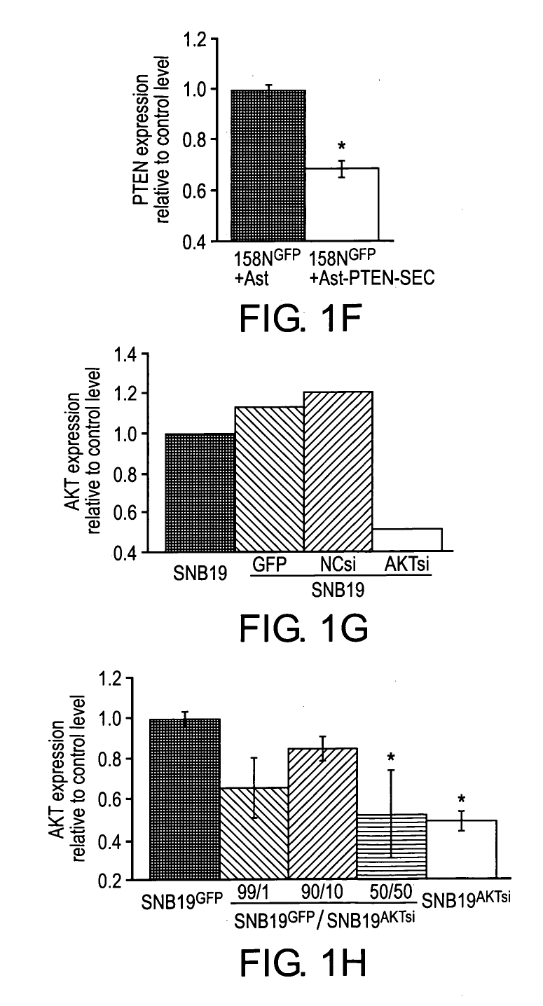

Referring now to the Drawings, FIG. 1 illustrates that PTEN or AKT silencing can be transmitted among cultured mammalian cells. FIG. 1(A) is a schematic representation of constructs used for cell transfection. A-I, GFP expression vector; A-II, DNA based siRNA vector; A-III, Silencing expression cassette; A-IIII, GFP expression cassette; PCMV: promoter of CMV, EGFP: enhanced GFP; PSV40: promoter of SV40; Hpt: bacterial hygromycin B phosphotransferase gene; SV40PA: SV40 polyadenylation signal sequence. PmU6; promoter of mouse U6 gene; PH1: promoter of human H1 gene; h-DNA: hairpin DNA. FIG. 1(B) shows PCR amplification of Hpt, GFP and SECs from genomic DNA isolated from stable transfected cell lines. 158N: non-transfected 158N cells; 158N^{GFP}: 158N cells stably transfected with GFP expression vector; 158N^{Psi}: 158N cells stably transfected with PTEN siRNA vector; 158N^{NCsi}: 158N cells stably transfected with non-specific negative control siRNA vector. FIG. 1(C) depicts Northern hybridization which indicates that PTEN is silenced in 158N^{Psi} cells compared to 158N cells or 158N^{GFP} cells. Top panel—Northern blot probed with ³²P labeled 18s cDNA probe; Bottom panel—the same blot probed with ³²P labeled PTEN cDNA probe. 158N, 158N^{GFP} and 158N^{Psi} are as described in FIG. 1B. FIG. 1(D) shows that PTEN silencing can be transmitted in 158N cells through co-culture. D-I, 158N^{GFP} cells and 158N^{Psi} cells co-cultured at different ratios as indicated. Top panel—cells viewed using phase contrast optics; Bottom panel—the same cells viewed under fluorescent illumination. D-II, RT-PCR analysis shows an obvious decrease of PTEN gene expression in co-cultures which initially contained only 1% PTEN silenced cells. The cells were analyzed after 3 days co-culture. D-III, Cells co-cultured for 3 days as in D-I and D-II were separated by FACS sorting. Sorted 158N^{GFP} cells were analyzed for PTEN mRNA expression by real time RT-PCR. The PTEN mRNA expression level in purified 158N^{GFP} cells was significantly decreased. PTEN mRNA level is shown as a ratio to control (158N^{GFP} cells). N=3; Bars indicate SEM; *p<0.01 versus control.

In FIG. 1(E), Northern hybridization analysis shows that PTEN silencing in PTEN-SEC transfected 158N cells can be transmitted to 158N^{NCsi} cells. Top panel—ethidium bromide stained gel for Northern blot; Bottom panel—Northern blot probed with ³²P labeled PTEN cDNA probe. PTEN-SEC: 158N cells 2 days after transfection with PTEN-SEC; 158N^{NCsi} 2d/8d: 158N^{NCsi} cells co-cultured with PTEN-SEC cells for 2 days, followed by 8 days hygromycin selection to eliminate PTEN-SEC cells; 158N^{NCsi} 2d/14d: 158N^{NCsi} cells co-cultured with PTEN-SEC cells for 2 days, followed by 8 days hygromycin selection and 6 more days culture. FIG. 1(F) depicts that PTEN silencing in primary astrocytes transfected with PTEN-SEC was transmitted to 158N^{GFP} cells. 158N^{GFP}+Ast: 158N^{GFP} cells co-cultured with astrocytes for 2 days, followed by elimination of astrocytes by 6 days of hygromycin selection; 158N^{GFP}+Ast-PTEN-SEC: 158N^{GFP} cells co-cultured with PTEN-SEC transfected astrocytes for 2 days, followed by elimination of astrocytes by 6 days of hygromycin selection. The remaining cells were analyzed for PTEN mRNA expression by real time RT-PCR. PTEN mRNA level is shown as a ratio to control (158N^{GFP}+Ast). N=3; Bars indicate SEM; *p<0.05 versus control. FIG. 1(G) shows that AKT mRNA expression was decreased in SNB19 cells stably transfected with AKT siRNA vector. Stable transfected cell lines were selected and analyzed for AKT mRNA expression by real time RT-PCR. AKT level is shown as a ratio to control (non-transfected SNB19 cells). SNB19: non-transfected SNB19 cells; SNB19^{GFP}: SNB19 cells stably transfected with GFP expression vector; SNB19^{NCsi}: SNB19 cells stably transfected with negative control siRNA vector; SNB19^{AKTsi}: SNB19 cells stably transfected with AKT siRNA vector. FIG. 1(H) illustrates that the AKT mRNA expression level in FACS sorted SNB19^{GFP} cells was decreased after co-culture with SNB19^{AKTsi} cells. SNB19^{GFP} cells and SNB19^{AKTsi} cells were co cultured for 3 days at different ratios as indicated, then separated by FACS sorting. Sorted cells were analyzed for AKT mRNA expression by real time RT-PCR. AKT mRNA level is shown as a ratio to control (SNB19^{GFP} cells). N=3; Bars indicate SEM; *p<0.05 versus control.

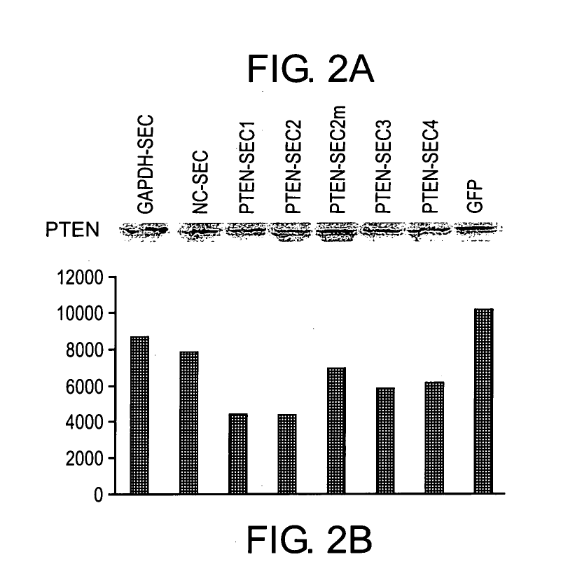

FIG. 2 shows testing the efficacy of PTEN-SECs. FIG. 2(A) illustrates Western blot analysis of PTEN expression in astrocytes after 2 days transfection with GAPDH-SEC, non-specific negative control SEC (NC-SEC), four PTEN-SECs, one mutated PTEN-SEC (PTEN-SEC-2m) and a GFP expression vector (GFP). In FIG. 2(B), the comparitive intensity of bands in (A) shows efficient silencing of PTEN expression by PTEN-SEC1 and PTEN-SEC2.

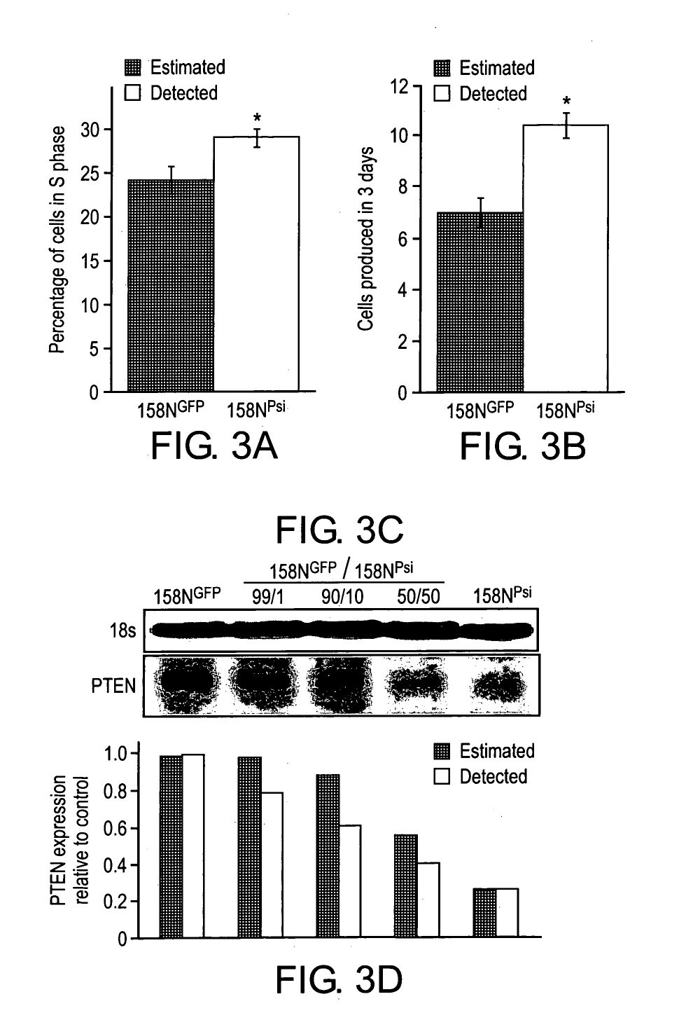

In FIG. 3(A), cell cycle analysis shows that the percentage of cells in S phase in 158N^{Psi} cells is higher than that of 158N^{GFP} cells. N=3; Bars indicate SEM; *p<0.01. In FIG. 3(B), cell profileration data shows that 158N^{Psi} cells proliferate faster than 158N^{GFP} cells. The relative rate of cell production was calculated as the final cell number divided by the initial cell number after 3 days culture. N=3; Bars indicate SEM; *p<0.001. FIG. 3(C) shows Northern blot analysis of PTEN expression after 3 days co-culture. Top panel—the blot probed with ³²P labeled 18s cDNA probe; Bottom panel—the same blot probed with ³²P labeled PTEN cDNA probe. (D) Comparison of PTEN expression levels detected from (C) and PTEN expression levels estimated after correction for faster proliferation rate of 158N^{Psi} cells as shown in (B). Expected values of PTEN mRNA levels were calculated based on the formula (6.97X+10.37Y*0.27)/(6.97 X+10.37Y) using values from (B) and (C). X and Y represent initial percentages of each cell type in the co-cultures, and 0.27 is the ratio of PTEN expression levels in 158N^{Psi} cells relative to 158N^{GFP} cells detected in (C).

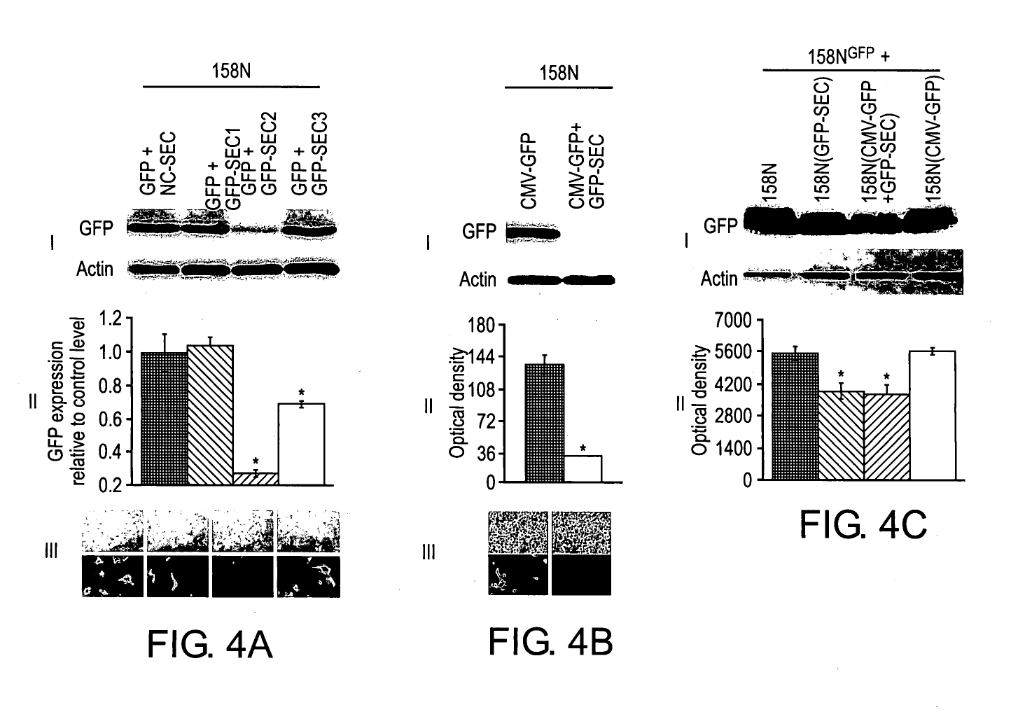

FIG. 4 shows that GFP silencing can be transmitted among cultured mammalian cells. FIG. 4(A) depicts testing the efficacy of GFP-SECs. A-I, Western blot analysis of GFP protein in 158N cells after 2 days co-transfection with GFP expression plasmid and Negative control silencing expression cassette (NC-SEC) or three different GFP silencing expression cassettes (GEP-SEC1, 2, 3). 5 μg of total protein was loaded in each lane. The blot was first probed with GFP antibody and then the same blot was re-probed with actin antibody. A-II, Real time RT-PCR analysis of GFP expression in cells as described in A-I. GFP level is shown as a ratio to control (159N cells co-transfected with GFP+NC-SEC). N=3; Bars indicate SEM; *p<0.01 versus control). A-III, Phase contrast (upper) and fluorescent (lower) photographs of cells described above. In FIG. 4(B), Western blot analysis shows that GFP was silenced in 158N cells co-transfected with CMV-GFP and GFP-SEC compared to 158N cells transfected with CMV-GFP only. Cells were harvested 6 days after transfection. B-I, Top panel—blot probed with GFP antibody; Bottom panel—the same blot re-probed with actin antibody. 10 μg of total protein per lane. B-II, Comparison of signal intensity in Western blots from B-I. N=3; Bars indicate SEM; *p<0.001. B-Ill, Phase contrast (upper) and fluorescent (lower) photographs of cells described above.

FIG. 4(C) illustrates the transmission of GFP silencing. C-I: Western blot analysis of GFP expression, 0.5 μg total protein loaded per lane. Top panel—Western blot probed with GFP antibody; Bottom panel—the same blot re-probed with actin antibody. 158N^{GFP} cells were co-cultured with transiently transfected 158N cells for 2 days, followed by 5 days hygromycin selection. The remaining 158N^{GFP} cells were analyzed for GFP expression. The bands are intense due to high levels of GFP expression, but were not saturated according to the readout of the imaging system. Lane 1: 158N^{GFP} cells co-cultured with non-transfected 158N cells; lane 2: 158N^{GFP} cells co-cultured with 158N cells transiently transfected with GFP-SEC; lane 3: 158N^{GFP} cells co-cultured with 158N cells co-transfected with CMV-GFP and GFP-SEC; lane 4: 158N^{GFP} cells co-cultured with 158N cells transfected with CMVGFP. C-II: Comparison of Western blot signal intensity in C-I. N=3; bars indicate SEM; * indicates lane 2 and lane 3 are different from lane 1 and lane 4, p<0.05.

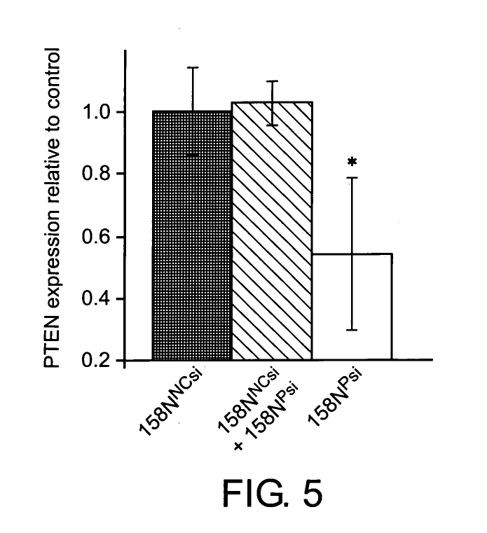

FIG. 5 shows real time RT-PCR analysis of PTEN expression in co-cultures from transwell plates. The data indicate that transmission of RNA silencing requires close proximity or direct cell contact. 158N^{NCsi} and 158N^{Psi} cells were co-cultured in transwell plates or cultured separately. After 3 days of co-culture, the cells in the well were harvested and analyzed for PTEN expression by real time RT-PCR. 158N^{NCsi}: 158N^{NCsi} cells were cultured both in the transwell plate and insert; 158N^{NCsi}+158N^{Psi}: 158N^{NCsi} cells were cultured in the plate with 158N^{Psi} cells in the transwell insert; 158N^{Psi}: 158N^{Psi} cells were cultured both in the transwell plate and insert. PTEN mRNA level is shown as a ratio to control (158N^{NCsi} cells). N=3; Bars indicate SEM; *p<0.05 versus control.

### Example 1

We used two cell lines (158N cells (Feutz et al., Glia, 34:241-252, 2001) derived from mouse oligodendrocytes and SNB19 cells (Mohanam et al., Oncogene, 14:1351-1359, 1997) derived from human glioblastoma cells) and mouse primary astrocytes to test whether the transmission of siRNA induced RNA silencing can occur among mammalian cells. A GFP expression vector (FIG. 1A-I), DNA based siRNA vectors for PTEN (using mouse U6 promoter), AKT (using human H1 promoter) or non-specific negative control (using either mouse U6 or human H1 promoter) (FIG. 1A-II), silencing expression cassettes (SEC) for PTEN and GFP (FIG. 1A-III) and GFP expression cassette (FIG. 1A-IIII) were prepared.

The efficacy of PTEN-SECs was tested (FIG. 2), and the most effective was used for further experiments. Stable, transfected cell lines were obtained by separate transfection of a GFP expression vector (GFP) and siRNA vectors for PTEN (Psi) or negative control (NCsi) into 158N cells (FIG. 1B). PTEN transcription level was decreased in the PTEN silenced cell line (158N^{Psi}) compared to non-transfected cells (158N) or to the cell line stably transfected with GFP expression vector (158N^{GFP}) (FIG. 1C). 158N^{GFP} cells and 158N^{Psi} cells were cultured separately or co-cultured at different ratios for 3 days (FIG. 1D-I). RT-PCR showed an obvious decrease of PTEN expression in co cultures with only 1% of 158N^{Psi} cells (FIG. 1D-II). PTEN mRNA detected by Northern hybridization decreased more than expected taking into account the higher proliferation of 158N^{Psi} cells (FIG. 3), suggesting transmission of a silencing signal between 158N^{GFP} and 158N^{Psi} cells. To confirm above results, the 158N^{GFP} cells were also separated from above co-cultures using FACS sorting. The purity of sorted 158N^{GFP} cells was confirmed by fluorescent analysis after plating, and cells were analyzed for PTEN expression by real time RT-PCR. The PTEN expression level in 158N^{GFP} cells which were co-cultured with 158N^{Psi} cells was significantly decreased compared to control 158N^{GFP} cells (FIG. 1D-III). Because of the absence of 158N^{Psi} cells, the silencing signal must have been transmitted from 158N^{Psi} cells to 158N^{GFP} cells through co-culture. PTEN-SEC (FIG. 1A-III) was transfected into 158N cells and co-cultured with 158N^{NCsi} for 2 days and then eliminated from co-cultures by 8 days hygromycin selection. The PTEN expression in remaining 158N^{NCsi} cells was decreased compared to 158N cells and control 158N^{NCsi} cells (FIG. 1E). Since PTEN silencing in 158N^{NCsi} was detected after elimination of 158N cells transfected with PTEN-SEC, it is clear that the siRNA induced PTEN silencing in PTEN-SEC cells was transmitted to 158N^{NCsi} cells by co-culture. PTEN mRNA levels in the silenced 158N^{NCsi} cells returned to control levels after 6 more days in culture (FIG. 1E), suggesting that the silencing signal was not amplified. Using the same approach, we also found PTEN RNA silencing transmission between PTEN-SEC transfected primary astrocytes from murine cerebral cortex and 158N^{GFP} cells (FIG. 1F).

Since PTEN silencing activates AKT and positively regulates cell proliferation, cells may more readily transmit silencing signals for PTEN than for other genes. Thus, we also tested silencing transmission for the AKT gene using a different cell type. SNB19 cells were transfected with GFP expression vector, AKT siRNA vector and negative control siRNA vector, and stable cell lines were obtained. AKT expression was decreased in cells transfected with AKT siRNA vector (SNB19^{AKTsi}) compared to either non-transfected cells (SNB19), cells transfected with a GFP expression vector (SNB19^{GFP}) or cells transfected with negative control siRNA vector (SNB19^{NCsi}) (FIG. 1G). The SNB19^{GFP} cells and SNB19^{AKTsi} cells were co-cultured at different ratios for 3 days and then separated by FACS sorting. The sorted SNB19^{GFP} cells were then analyzed for AKT expression by real-time RT-PCR. AKT expression was significantly decreased in SNB19^{GFP} cells co-cultured with SNB19^{AKTsi} cells compared to non co-cultured SNB19^{GFP} cells (FIG. 1H). We did not find transmission of RNA silencing between cells in transwell cultures, suggesting that transmission requires close proximity or direct cell-cell contact. See FIG. 5.

### Example 2

PTEN and AKT are endogenous genes with profound, yet opposite effects on cell survival and growth. To test whether silencing transmission could also occur for a foreign gene that does not regulate cell cycle/growth we examined whether GFP could be silenced in 158N^{GFP} cells through RNA silencing transmission. Three GFP-SECs (FIG. 1A-III) were made and tested for efficacy of GFP silencing by Western blot (FIG. 4A-I), real time RT-PCR (FIG. 4A-II) and the intensity of fluorescence (FIG. 4A-III). The most effective GFP-SEC (GFP-SEC2), a CMV-GFP cassette (FIG. 1A-IIII), or both, were transiently transfected into 158N cells. The GFP was silenced in 158N cells that were co-transfected with CMV-GFP and GFP-SEC compared to 158N cells transfected with CMV-GFP only (FIG. 4B-I, 4B-II, 4B-III). The transfected cells were then co-cultured with 158N^{GFP} cells for 2 days, followed by 5 days of selection in hygromycin. (In parallel experiments, the same hygromycin treatment eliminated all cells that were not hygromycin resistant.) GFP protein was decreased significantly in 158N^{GFP} cells that were co-cultured with 158N cells transfected with GFP-SEC alone or co-transfected with GFP-SEC+CMV-GFP, as compared to 158N^{GFP} cells that were co-cultured either with 158N cells or 158N cells transfected with CMV-GFP (FIGS. 4C-I, 4C-II). This result indicates transmission of a silencing signal from GFP-SEC transfected 158N cells to 158N^{GFP} cells.

Since there is no GFP mRNA target in 158N cells, the silencing signal formed in GFP-SEC transfected 158N cells is likely related to the siRNA itself. Evidence against the ability of siRNAs to act as mobile silencing signals comes from a study where the C. elegans rde-4 mutation, which interferes with production of siRNAs, did not interfere with systemic silencing (Tabara et al., Cell, 199:123-132, 1999). However, it is possible that different mobile silencing signals function in different types of cells. Alternatively, if redundant pathways exist then small amounts of siRNAs may be produced even when the dominant pathway is disrupted. The level of silencing transmission observed here for GFP is less than that seen for PTEN in FIG. 1. Silencing transmission may not be equally efficient, even among genes that are normally expressed. GFP is a foreign protein, it is expressed at extremely high levels in the transfected cells, and we used transiently transfected cells instead of stable cell lines to generate the silencing signal. Given these experimental differences, it is not surprising that the level of silencing transmission differs between GFP and PTEN.

Our results show that transmission of siRNA induced silencing can occur in mammalian cells, and is similar to the phenomena observed in plant cells where siRNA induced silencing can be transmitted to 10-15 neighboring cells without siRNA amplification (Himber et al., EMBO J., 22:4523-4533, 2003). The mechanisms of RNA silencing transmission remain unknown either in plant or animal cells. RNA silencing transmission in mammalian cells is of great interest at both fundamental and applied levels. It provides a novel pathway by which individual cells can suppress the expression of genes in neighboring cells. Co-culture with silenced cells also offers an alternative approach for experimental silencing of genes in cells which are difficult to transfect. Importantly, RNA silencing transmission can be used to facilitate therapeutic strategies. One particular advantage is that DNA based siRNA constructs can be transfected into cells taken directly from individual patients. After replacement, such cells could act as well localized siRNA resources for daughter cells and/or neighboring cells in vivo. Thus, even if mammalian cells do not normally utilize RNA silencing transmission, it can be a potent tool for experimental and therapeutic manipulation of gene expression.

All references cited in this disclosure are incorporated by reference in their entirety.

While the invention has been described in detail and with reference to specific embodiments thereof, it will be apparent to one skilled in the art that various variations and modifications can be made therein without departing from the spirit and scope thereof. All such variations and modifications are intended to be included within the scope of this disclosure and the present invention and protected by the following claims.

We claim:

1. A method of reducing expression of a target gene in a mammalian cell, comprising:
a) introducing siRNA into a first mammalian cell such that expression of the target gene is reduced in the first mammalian cell, and
b) exposing a second mammalian cell which does not contain siRNA that reduces expression of the target gene to the siRNA-containing first mammalian cell, thereby resulting in reduced expression of the target gene in the second mammalian cell.

2. The method of claim 1 wherein introducing siRNA into the first mammalian cell is by transfection with an expression vector which directs expression of siRNA for the target gene such that expression of the target gene is reduced in the first mammalian cell.

3. The method of claim 1 wherein the first or second mammalian cell is a nervous cell.

4. The method of claim 3 wherein the nervous cell is a neuronal cell.

5. The method of claim 3 wherein the nervous cell is a glial cell.

6. The method of claim 5 wherein the glial cell is an astrocyte.

7. The method of claim 5 wherein the glial cell is an oligodendrocyte.

8. The method of claim 1 wherein the target gene is a mutant gene which causes a genetic disease.

9. The method of claim 8 wherein the genetic disease is Huntington's disease.

10. The method of claim 8 wherein the genetic disease is Pelizaeus-Merzbacher disease.

11. The method of claim 1 wherein the target gene is a gene whose overexpression causes a disease.

12. The method of claim 11 wherein the disease is a cancer.

13. The method of claim 12 wherein the cancer is one in which the EGF-receptor is overexpressed.

14. The method of claim 11 wherein the disease is Pelizaeus-Merzbacher disease in which the proteolipid protein gene is overexpressed.

15. The method of claim 1 wherein the target gene is a death gene which causes loss of a cell after a traumatic injury.

16. The method of claim 1 wherein the target gene is an MHC gene whose absence increases the vulnerability of a tumor cell to NK mediated killing.

17. The method of claim 1 wherein the target gene is a gene which promotes cell survival such that expression of the siRNA induces cell death.

18. The method of claim 17 wherein the cell death occurs in a tumor cell or an astroglial scar.

19. A method of reducing expression of a target gene in a mammal, comprising:
a) introducing siRNA into a first mammalian cell such that expression of the target gene is reduced in the first mammalian cell, and
b) introducing the first mammalian cell containing the siRNA into the mammal whereby expression of the target gene is reduced in a cell of the mammal.

20. The method of claim 19 wherein introducing siRNA into the first mammalian cell is by transfection with an expression vector which directs expression of siRNA for the target gene such that expression of the target gene is reduced in the first mammalian cell.

21. The method of claim 19 wherein the first mammalian cell is a nervous cell and the cell of the mammal is a nervous cell.

22. The method of claim 21 wherein the nervous cell is a neuronal cell.

23. The method of claim 21 wherein the nervous cell is a glial cell.

24. The method of claim 23 wherein the glial cell is an astrocyte.

25. The method of claim 23 wherein the glial cell is an oligodendrocyte.

26. The method of claim 19 wherein the target gene is a gene whose overexpression causes a disease.

27. The method of claim 26 wherein the disease is a glioblastoma or other cancer.

28. A transfected mammalian cell wherein the cell is transfected with an expression vector which directs expression of siRNA for a target gene such that expression of the target gene is reduced in the transfected mammalian cell, the siRNA expression level being sufficient to signal reduction of target gene expression in a mammalian cell not transfected with siRNA for the target gene.

29. The transfected cell of claim 28 which is a nervous cell.

30. The transfected cell of claim 29 wherein the nervous cell is a neuronal cell.

31. The transfected cell of claim 29 wherein the nervous cell is a glial cell.

32. The transfected cell of claim 29 wherein the glial cell is an astrocyte.

33. The transfected cell of claim 29 wherein the glial cell is an oligodendrocyte.
